(19) United States
(12) Patent Application Publication
(10) Pub. No.: US 2022/0049216 A1
(43) Pub. Date: Feb. 17, 2022

(54) MANUFACTURE OF STRUCTURE CAPABLE OF FORMING THREE-DIMENSIONAL NEURONAL SPHEROID AND GENERATING NEURITE THROUGH VARIOUS SURFACE PROCESSES

(71) Applicant: Korea University Research and Business Foundation, Seoul (KR)

(72) Inventors: Dong Hwee KIM, Seoul (KR); Geon Hui LEE, Samcheok-si (KR)

(73) Assignee: Korea University Research and Business Foundation, Seoul (KR)

(21) Appl. No.: 17/413,760

(22) PCT Filed: Dec. 24, 2019
(86) PCT No.: PCT/KR2019/018382

§ 371 Date: Jun. 14, 2021

(30) Foreign Application Priority Data
Dec. 24, 2018 (KR) 10-2018-0168191

(51) Int. Cl. C12N 5/0793; C12N 5/00; G01N 33/50

(52) U.S. Cl. CPC C12N 5/0619; C12N 5/0068; C12N 5/0062; G01N 33/5058; G01N 2800/28; C12N 2503/02; C12N 2533/30; C12N 2535/00; C12N 2533/20; C12N 2513/00

(57) ABSTRACT

The present invention relates to the manufacture of a platform for forming a neuronal spheroid and, more specifically, to the manufacture of a structure capable of simultaneously forming a test-tube three-dimensional neuronal spheroid and generating a neurite by forming a three-dimensional neuronal spheroid with a micro-platform and forming a neurite between the neuronal spheroid and the micro-platform so as to enable signal transduction, which is an essential function of a nerve cell.

### TECHNICAL FIELD

The present invention relates to a platform for forming a three-dimensional neuronal spheroid and neurite.

### BACKGROUND ART

Organs and tissues in the human body are basically composed of cells along with the extracellular matrix. With the recent publication of the research results showing that three-dimensionally (3D) cultured cells cultured in a three-dimensional fashion elicit similar biological reactions in vivo, the significance of 3D cell culture techniques has come into the spotlight.

Meanwhile, in the case of a developmental process of nerve cells, stem cells have a pattern in which they differentiate into nerve cells, which finally form neurites so that the nerve cells can be connected to each other. The formation of neurites has a direct influence on the formation of nerve fasciculi and the development of nerve cells. It is known that the formation of the neurites is determined through the cell-cell interaction with the extracellular matrix.

The existing research on the formation of neurites was often conducted by inducing the formation of neurites using laminin, poly-L-lysine, or poly-D-lysine. For example, there has been a lot of research conducted to coat nanofibers with laminin or poly-L-lysine to improve an adhesive force between the cells and a bottom, which makes it possible to enhance the viability of nerve cells and help the growth of neurites. However, all the results of this research were obtained from two-dimensional cell culture, which is far from what actually occurs three-dimensionally. Meanwhile, when the nerve cells are cultured in a platform so that the nerve cells can be cultured in a three-dimensional fashion, it has a drawback in that the shape of the neuronal spheroids is not uniform or the neurites do not grow.

### RELATED-ART DOCUMENT

### Patent Document

Korean Patent Laid-Open Publication No. 10-2017-0029237

### DISCLOSURE

### Technical Problem

When an interaction force between cells and a structure is stronger than an interaction force between cells, nerve cells can differentiate to form neurites, but a spherical 3D cell aggregate having a uniform shape is not formed. On the other hand, when the interaction force between the cells and the structure is weaker than the interaction force between the cells, the nerve cells differentiate to form a spherical 3D cell aggregate having a uniform shape, but neurite formation is not possible. To screen drugs at the test tube level or mimic a neuronal spheroid in vivo to study biological similarity, a neuronal spheroid should be formed in a three-dimensional fashion and neurites should be simultaneously formed. Therefore, in the present invention, there is a need for development of a micro-platform capable of optimizing an interaction force between cells and a structure to generate neurites as well form 3D neuronal spheroids having a uniform shape.

### Technical Solution

To solve the above problems, the present invention is directed to providing a micro-platform optimizing an interaction force between cells and a structure to generate neurites as well form 3D neuronal spheroids having a uniform shape.

Specifically, the present invention provides a platform for culturing 3D nerve cells, which includes a substrate consisting of concave microwells surface-treated with 3-aminopropyltriethoxysilane (APTES).

Each of the concave microwells may refer to a recessed structure in which nerve cells may be fixed and cultured on a flat substrate. The concave microwells may be used without limitations to size and shape as long as a space sufficient to form one neuronal spheroid can be provided to nerve cells during culture. In one specific embodiment, the concave microwells were used by manufacturing a substrate in which hemispherical concave microwells having a diameter in a range of 100 μm to 1,000 μm were formed. To form spherical neuronal spheroids having a uniform size, a hemispherical shape was selected as a structure capable of uniformly interacting between cells and a culture platform.

When a platform used to culture 3D nerve cells has a diameter of 100 μm or less, the size of the formed neuronal spheroids is small, which makes the 3D cell culture meaningless. On the other hand, because the size of the neuronal spheroids formed using the nerve cells having a size of 1,000 μm or more is greater than 300 μm, cell death (necrosis) may occur inside the neuronal spheroid due to the lack of oxygen and nutrients. Therefore, a hemispherical platform having a diameter of 100 to 1,000 μm is used in the present invention.

The substrate may be made of polydimethylsiloxane (PDMS) having excellent biocompatibility.

The present inventors have found that neuronal spheroids having a uniform spherical shape are formed as nerve cells are cultured on a substrate in which concave microwells surface-treated with 3-aminopropyltriethoxysilane (APTES) are formed, and also found that formation of neurites is promoted.

A nerve cell is composed of a neuronal spheroid and neurites, and may form a network between other nerve cells through the neurites to rapidly transmit and receive signals.

In one specific embodiment of the present invention, the substrate surface-treated with 3-aminopropyltriethoxysilane (APTES) may be further surface-treated with one or more of carbon nanotubes (CNTs), laminin, and poly-L-lysine (PLL).

The nerve cells were cultured in a platform for culturing 3D nerve cells in which the substrate surface-treated with 3-aminopropyltriethoxysilane (APTES) is further surface-treated with one of carbon nanotubes (CNTs), laminin, and poly-L-lysine (PLL). As a result, it was confirmed that there is no difference in forming neuronal spheroids when the substrate is further surface-treated with carbon nanotubes. Also, it was confirmed that formation of neurites is promoted when the substrate is further surface-treated with laminin or poly-L-lysine. It was confirmed that the formation of the neuronal spheroids and neurites may be artificially adjusted by adjusting an interaction force between the nerve cells and the platform by further treating the substrate with the substance(s).

In another specific embodiment, a plurality of concave microwells may be formed in various patterns in the substrate. One nerve cell may be cultured in one concave microwell, and open spaces may be present between the adjacent concave microwells in the case of the plurality of concave microwells. Neurites formed in the nerve cell cultured in one concave microwell are connected to neuronal spheroids of the nerve cells cultured in adjacent concave microwells by means of the open spaces to form one neural network in vitro. In this way, the concave microwells may be used to study a signal transduction mechanism between nerve cells and screen drugs for treatment of neurological disorders by mimicking a 3D environment similar to an in vivo neural network environment in vitro.

The present invention provides a method of culturing 3D nerve cells, which includes culturing nerve cells in the above-described platform for culturing 3D nerve cells.

The present invention provides a method of screening a drug for treatment of damaged nerves or neurological disorders, which includes the following steps:

culturing nerve cells in the platform for culturing 3D nerve cells according to the present invention;

treating the cultured nerve cells with a candidate substance;

treating the nerve cells treated with the candidate substance with a substance that causes damaged nerves or neurological disorders; and

comparing a degree of formation of neurites and neuronal spheroids in the nerve cells with that of the control (not treated with the candidate substance).

The candidate substance may be any one selected from the group consisting of a peptide, a protein, a non-peptidic compound, a synthetic compound, a fermentation product, a cell extract, a plant extract, and an animal tissue extract, but the present invention is not limited thereto.

The candidate substance may be a single compound, a mixture of compounds (for example, a natural extract or a cell or tissue culture), an antibody, or a peptide, or may be obtained from a library of synthetic or natural compounds. Methods of obtaining such a library of compounds are known in the art. A library of synthetic compounds is commercially available from Maybridge Chemical Co. (UK), Comgenex (USA), Brandon Associates (USA), Microsource (USA), and Sigma Aldrich (USA), and a library of natural compounds is commercially available from Pan Laboratories (USA) and MycoSearch (USA).

In the present invention, the damaged nerves or neurological disorders may include any one of a brain damage, a brain disease, a spinal cord injury, a peripheral nerve injury, a peripheral nerve disorder, or amyotrophic lateral sclerosis. In this case, the brain damage or brain disease may include any one of dementia, Parkinson's disease, Alzheimer's disease, Huntington's disease, epilepsy, stroke, palsy, an ischemic brain disease, or a degenerative brain disease.

The contents overlapping with the above-described contents regarding the platform for culturing 3D nerve cells will be omitted.

### Advantageous Effects

First, a micro-platform can be used to culture 3D neuronal spheroids having a uniform shape.

Second, an interaction force between cells and a structure can be adjusted to perform a surface process so that the structure can have physically, chemically and structurally different properties.

Third, the neuronal spheroids can be aligned. When a network is designed in concave wells, neurites of the neuronal spheroids generated by the surface process can be interconnected to align the neuronal spheroids in a desired fashion.

Fourth, the neuronal spheroids can be used to screen drugs by effectively mimicking in vivo nerve cells.

### DESCRIPTION OF DRAWINGS

FIG. 1 is a schematic diagram showing a process of surface-treating a concave microwell in which a neurite and a neuronal spheroid may be formed.

FIG. 2 shows different chemical properties of five surfaces obtained by performing a surface process using Fourier transform infrared spectroscopy.

FIG. 3 shows surface properties of concave microwells obtained by five surface processes using an atomic force microscope. Scale bar: 500 nm

FIG. 4 shows different physical properties of the concave microwells obtained by five surface processes by measuring a contact angle.

FIG. 5 shows different cell adhesive forces of the concave microwells obtained by five surface processes.

FIG. 6 shows that neuronal spheroids are formed in the concave microwells by five surface processes: A) Optical microscope image obtained by date; B) Days required to form neuronal spheroids; and C) Size of neuronal spheroids. Scale bar: 100 μm

FIG. 7 shows an image of transporting calcium in the neuronal spheroids cultured on the five surfaces using a calcium indicator (Fluo-4 AM). Scale bar: 50 μm

FIG. 8 shows whether the cultured neuronal spheroids are formed and neurites are generated in the manufactured concave microwells by the five surface processes: A) Electron microscope image of neuronal spheroids; B) Image of fluorescence-stained neuronal spheroids and neurites; C) Number of neurites formed in each concave microwell; D) Length of neurites formed in each concave microwell; E) Relationship between contact angle and cell adhesive force and formation of nerve cells; and F) Schematic diagram showing the relationship between each surface and adhesive force of nerve cells and formation of neurites. Scale bar: 200 μm

FIG. 9 is a microscope image showing the neurites connected to the neuronal spheroids cultured in a network structure in a uniform distance using a surface process. Scale bar: 500 μm

FIG. 10 shows a neurite reduction model for administering amyloid-beta to verify a drug screening model: A) Neuronal spheroids in concave microwells treated with APTES; B) Accumulation of amyloid-beta; C) Neuronal spheroids treated with amyloid-beta; D) Increased accumulation of amyloid-beta; E) Comparison of accumulation of amyloid-beta; F) Comparison of the number of neurites; and G) Comparison of lengths of neurites.

### BEST MODE

Hereinafter, the present invention will be described in further detail with reference to Examples according to the present invention. However, it should be understood that the following Examples are not intended to limit the scope of the present invention.

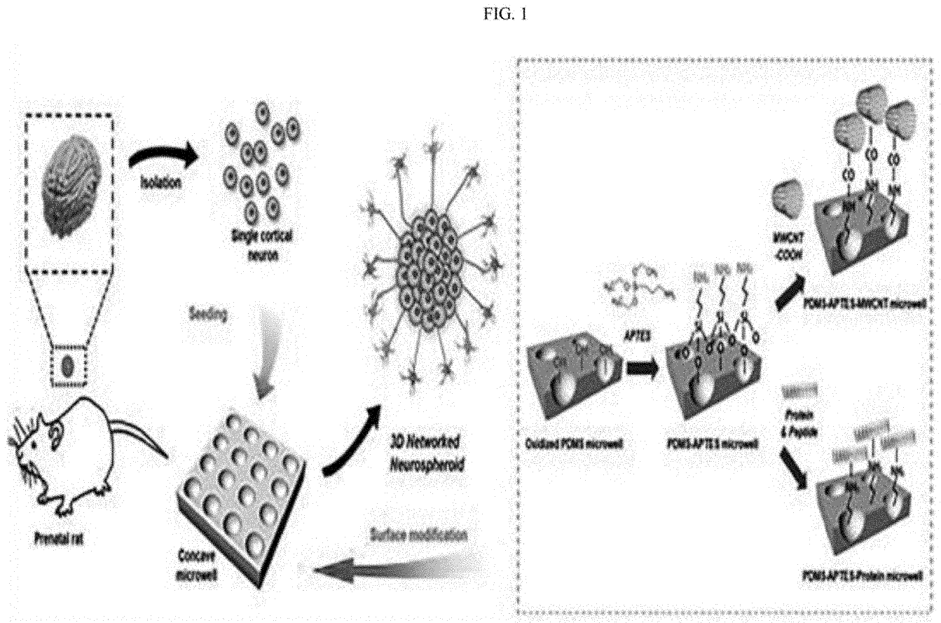

An interaction force between cells and a neurite structure was optimized by a surface process so that a neurite structure could be generated and at the same time, neuronal spheroids could be formed in a regular shape and at regular intervals. Then, nerve cells extracted from a prenatal rat were cultured in concave microwells made of polydimethylsiloxane (PDMS) (FIG. 1).

### [Example 1] Surface Process

Surfaces of concave microwells were processed using an aminosilane functional group of APTES, carbon nanotubes, poly-L-lysine, and laminin. First, the concave microwells were surface-treated with oxygen plasma (100 W, 30 s), and then further surface-treated with a 10% (volume ratio relative to water) APTES solution or 0.01% (volume ratio relative to water) carbon nanotubes, or 20 μg/ml laminin or 20 μg/ml poly-L-lysine for approximately 1 hour.

### [Example 2] Confirmation of Chemical, Surface and Physical Properties of Surface

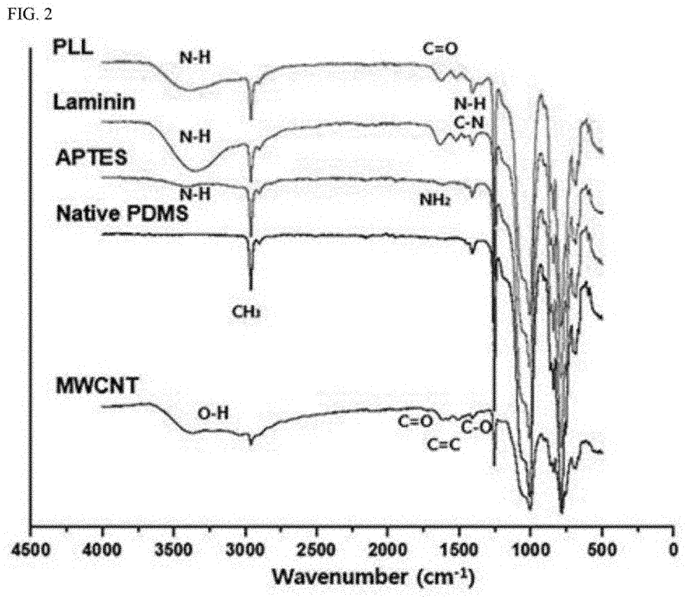

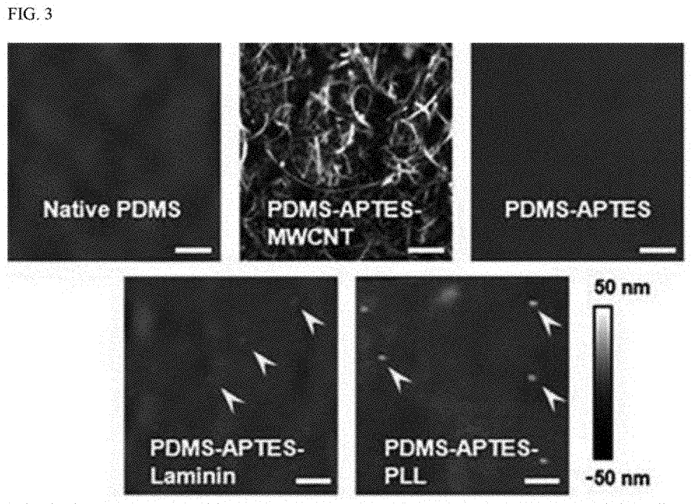

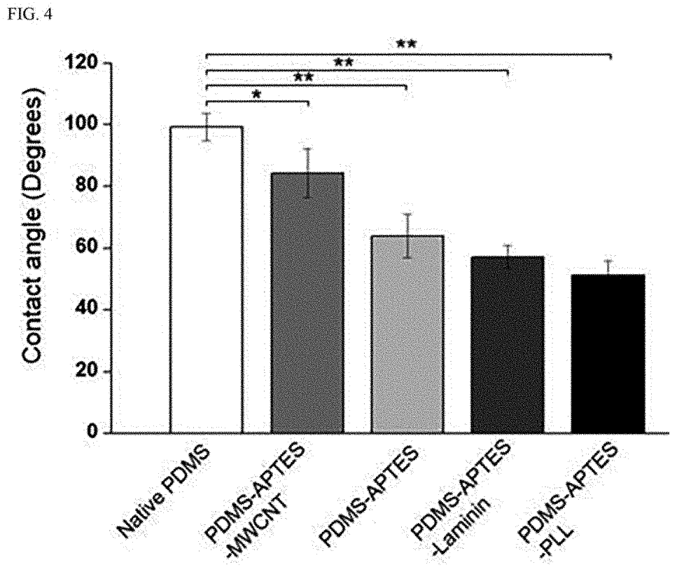

To determine a chemical difference in surfaces of concave microwells coated with several substances, it was confirmed that the five concave microwells showed different chemical properties using Fourier transform infrared (FTIR) spectroscopy (FIG. 2). Also, it was confirmed that the different substances were attached to the surfaces of the concave microwells using an atomic force microscope (AFM) (FIG. 3). In addition, it was confirmed that the highest contact angle was formed on a surface of polydimethylsiloxane itself and the smallest contact angle was formed on the surface treated with poly-L-lysine when the contact angle was measured (FIG. 4).

### [Example 3] Experiment for Attachment of Nerve Cells

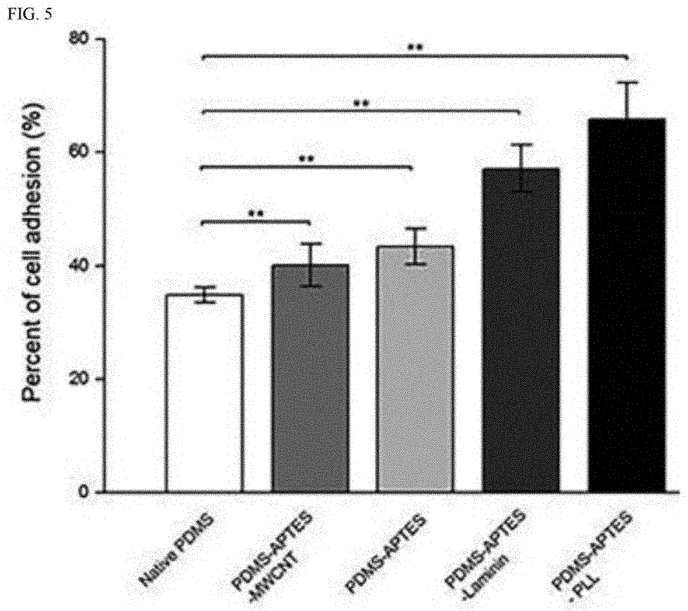

To measure an interaction force between the cells and the structure, an experiment for attachment of nerve cells to five surfaces having different properties, which were manufactured by a surface process, was performed. 2*10⁵ nerve cells were seeded under each surface condition, and cultured for 3 hours, and a culture medium was then replaced three times to remove non-adherent cells. To measure an amount of the remaining cells, the cells were quantified using Cell Counting Kit-8 (Dojindo, USA). As a result, it was confirmed that the smallest amount of cells were attached to the surface of polydimethylsiloxane itself, and the highest amount of cells were attached to the surface treated with poly-L-lysine (FIG. 5).

### [Example 4] Formation of Neuronal Spheroids

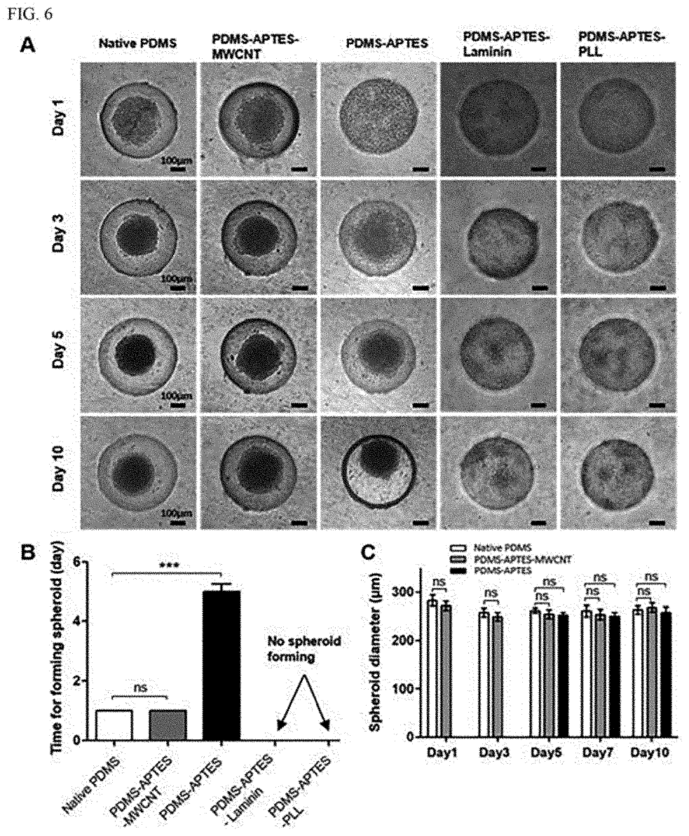

A formation period of the neuronal spheroids was determined under different surface conditions. The neuronal spheroids were formed within one day after cell seeding in the concave microwells made of polydimethylsiloxane and the concave microwells surface-treated with carbon nanotubes. It was shown that the neuronal spheroids were formed on day 5 after cell seeding in the case of the concave microwells treated with APTES, and the neuronal spheroids were not formed even after 10 days in the case of the concave microwells treated with laminin or poly-L-lysine. Meanwhile, it was confirmed that there was no significant difference in size between the generated neuronal spheroids (FIG. 6).

### [Example 5] Confirmation of Signal Transduction via Neurons

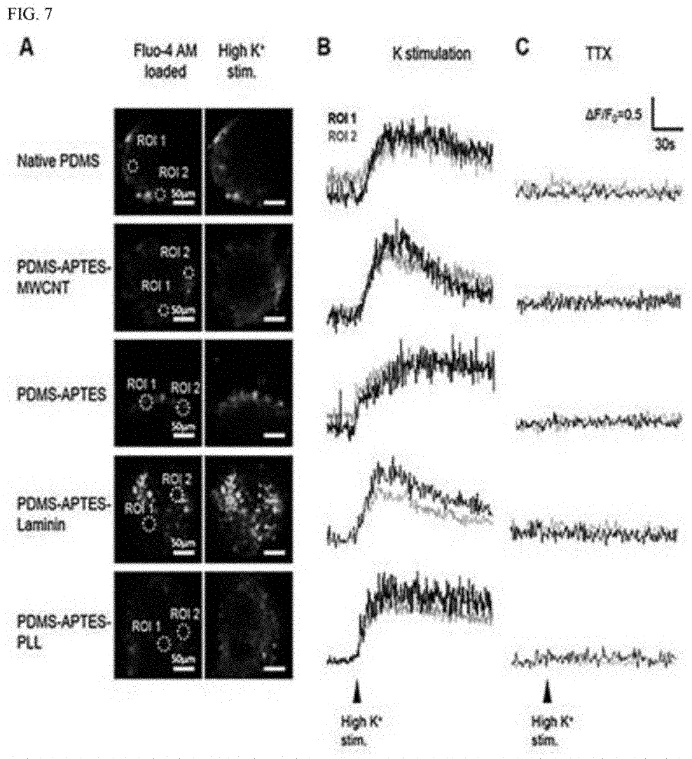

To verify the neurotransmission activated by potassium, signal transduction was measured in a fluorescence image using an intracellular calcium indicator Fluo-4 AM. The neuronal spheroids into which Fluo-4 was injected in advance were stimulated with a high concentration of potassium to confirm calcium transport. In this way, it was verified that abnormalities in the nerve cells grown on the surface under the five different conditions were not induced by the surface process when the nerve cells were subjected to the surface process (FIG. 7).

### [Example 6] Confirmation of Generation of Neurites and Formation of Neuronal Spheroids

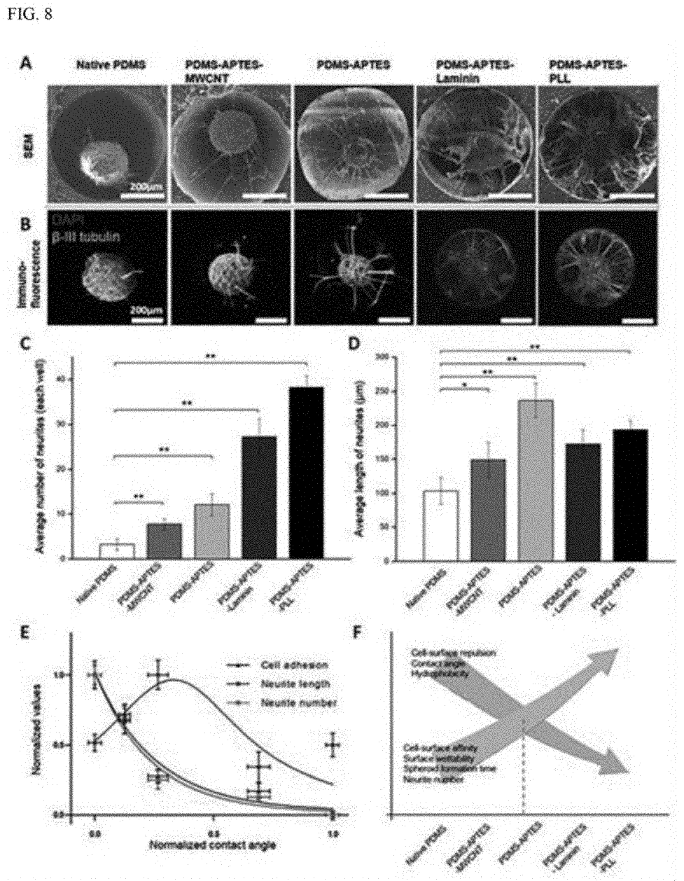

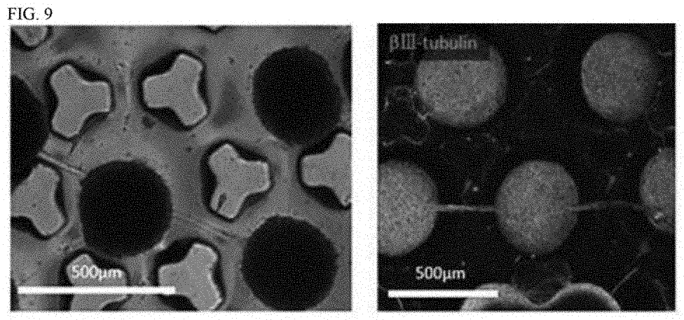

The nerve cells were incubated for 10 days in the concave microwells under the five different surface conditions, fluorescence-stained with β-III tubulin, and then observed with an electron microscope to determine whether the neuronal spheroids were formed and the neurites were generated. As a result, the neuronal spheroids were formed in the concave microwells made of polydimethylsiloxane and the concave microwells treated with carbon nanotubes and APTES. It was confirmed that the highest amount of neurites were formed in the concave microwells treated with poly-L-lysine, and the neurites were formed longest in the concave microwells treated with APTES. Therefore, it was concluded that it was most suitable to generate the neurites and form the neuronal spheroids in the concave microwells treated with APTES. Based on the results, it was confirmed that an repulsive force between the cells and the structure decreased, a contact angle decreased, hydrophobicity decreased, the affinity between cells and the structure increased, surface wettability increased, the time taken to form a cell structure increased, and the number of formed neurites increased in the order of the concave microwells made of polydimethylsiloxane, and the concave microwells treated with carbon nanotubes, APTES, laminin, and poly-L-lysine (FIG. 8). Also, it was confirmed that neurites between the neuronal spheroids were formed and connected through the network between the concave microwells treated with APTES (FIG. 9).

### [Example 7] Experiment for Administration of Amyloid-Beta for Drug Screening

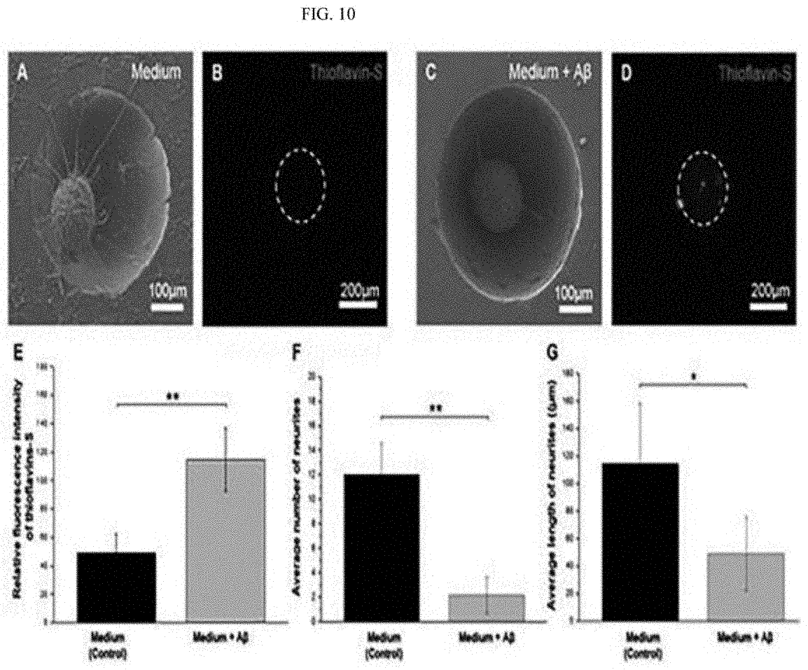

After confirming that it was optimal to form the neuronal spheroids and generate the neurites in the concave microwells treated with APTES, amyloid-beta that is known to be a representative substance that causes Alzheimer's disease was administered to determine whether the concave microwells treated with APTES can be used to screen drugs. To determine the accumulation of amyloid-beta into the neuronal spheroids, thioflavin-S was used to obtain a fluorescence image. As a result, it was confirmed that the number and length of the neurites in the neuronal spheroids treated with amyloid-beta decreased dramatically. Based on the results, it was verified that the method provided in the present invention is able to be used to screen drugs (FIG. 10).

1. A platform for three-dimensional (3D) nerve cells culture, comprising concave microwells surface-treated with 3-aminopropyltriethoxysilane (APTES).

2. The platform of claim 1, wherein the concave microwells are hemispherical with a diameter of 100 μm to 1,000 μm.

3. The platform of claim 1, wherein the platform for culturing 3D nerve cells promotes the formation of neuronal spheroids and neurites.

4. The platform of claim 1, wherein the substrate consists of a plurality of concave microwells, wherein open spaces are present between the adjacent concave microwells.

5. The platform of claim 1, wherein the substrate is further surface-treated with one or more of carbon nanotubes (CNTs), laminin, and poly-L-lysine (PLL).

6. The platform of claim 1, wherein the substrate consisting of the concave microwells is made of polydimethylsiloxane (PDMS).

7. A method of culturing 3D nerve cells in vitro, comprising:
culturing nerve cells in the platform for culturing 3D nerve cells defined in claim 1.

8. A method of screening a drug for treatment of damaged nerves or neurological disorders, comprising:
culturing nerve cells in the platform for culturing 3D nerve cells defined in claim 1;
treating the cultured nerve cells with a candidate substance;
treating the nerve cells treated with the candidate substance with a substance that causes neurological disorders; and
comparing a degree of formation of neurites and neuronal spheroids in the nerve cells with that of the control (not treated with the candidate substance).
